(19) United States
(12) Patent Application Publication
(10) Pub. No.: US 2022/0142688 A1
(43) Pub. Date: May 12, 2022

(54) VARIABLE ANGLE FIXATION MECHANISM, AND BONE PLATE AND BONE TREATMENT TOOL

(71) Applicant: MEIRA Corporation, Nagoya-shi (JP)

(72) Inventors: Naoto Tsubokawa, Niigata-shi (JP); Koji Moriya, Niigata-shi (JP); Takaharu Ishii, Minokamo-shi (JP)

(73) Assignee: MEIRA Corporation, Nagoya-shi (JP)

(21) Appl. No.: 17/453,969

(22) Filed: Nov. 8, 2021

(30) Foreign Application Priority Data
Nov. 11, 2020 (JP) 2020-188074

(51) Int. Cl. A61B 17/80

(52) U.S. Cl. CPC A61B 17/8014; A61B 17/8057

(57) ABSTRACT

A fixation mechanism in which a first thread groove extending in one turning direction and a second thread groove extending in a turning direction opposite to the one turning direction and intersecting the first thread groove are formed in a first member-side thread portion of a first member. The thread divided portion being formed as a result of the first thread groove and the second thread groove intersecting each other. A second member-side thread portion of a second member can be screwed to the first thread groove of the first member-side thread portion. The second member is fixed at a suitable angle 0 relative to the first member.

### TECHNICAL FIELD

The present invention relates to a fixation mechanism for fixing two members at a suitable angle, and a bone plate and a bone treatment tool that are used for treatment for fractured portions or the like and in which the fixation mechanism can be used.

### BACKGROUND ART

Many bone fragments that need to be rejoined may be generated due to fracturing of end portions (a distal end portion and a proximal end portion) of a bone or fracturing in the vicinities of the end portions. In treatment for such fractured portions, a bone plate that is attached to a bone fragment and a bone main body in the manner of a bridge is used to fix the bone fragment to the bone main body after restoring the bone fragment to its original position and orientation.

Such bone plates include those that have an opening for fixing a bone screw. As described in JP 2012-502687A and JP 2006-130317A, there is a bone plate in which an internal thread portion is provided on the inner surface of an opening for fixing a screw, and a bone screw (see FIG. 29 of JP 2012-502687A and FIG. 5 of JP 2006-130317A) that includes a head portion provided with an external thread portion that can be screwed to the internal thread portion is used for such a bone plate. Bone plates and bone screws that have internal thread portions and external thread portions as described above have an increased effect of fixation to a bone.

A bone plate and a bone screw such as those described above can be fixed to each other in a state where the longitudinal axis of the bone screw is aligned with the axis of the opening in the bone plate, but this angle (relative angle between the bone plate and the bone screw) may not be the optimum angle. For example, there are cases where an angle that is different from an originally set angle is desired, depending on the shape of the bone to which the bone plate is applied, a force that the bone is subjected to, or any other fixation state to be achieved.

Therefore, in recent years, there has been demand for a variable angle fixation mechanism (also called a “polyaxial locking mechanism”) with which the bone screw can be fixed to the bone plate while increasing the freedom of the insertion direction of the bone screw. When this mechanism is adopted, the bone screw can be inserted or fixed at a suitable angle and the bone fragment can be fixed at a desired position. For example, JP 2016-512711A and JP 2019-526375A disclose configurations for fixing the bone screw such that an angle relative to the bone plate is variable.

JP 2012-502687A (WO2010/030847A1), JP 2006-130317A (US2005/0065521A1), JP 2016-512711A (WO2014/160166A2), and JP 2019-526375A (WO2018/048668A1) are examples of related art.

### SUMMARY OF THE INVENTION

In the mechanisms described in JP 2016-512711A and JP 2019-526375A, a thread portion (thread ridge) formed in a hole of the plate is divided by at least one recess extending in the axial direction of the hole, and the bone screw is fixed at a suitable angle relative to the bone plate by engaging the divided thread portion and a thread portion formed in a head portion of the bone screw.

However, in the case of such a mechanism, in order to divide the thread portion formed in the hole of the plate, the recess needs to be processed using an axis different from that of the hole, which makes manufacturing troublesome. Also, in JP 2019-526375A, the thread portion formed in the hole of the plate is a tapered thread, and the processing of the thread portion is also troublesome.

Therefore, the present invention provides a fixation mechanism for fixing two members at a suitable or any angle, which can be manufactured easily, enables fine adjustment of the relative angle, and with which two members can be quickly and firmly fixed, and a bone plate and a bone treatment tool that are used to treat fractured portions or the like and in which the fixation mechanism can be used.

The above object can be achieved by the following.

A fixation mechanism that can fix a first member and a second member to each other at a suitable or any angle, wherein

the first member includes a base plate, a through hole that extends through the base plate, and a first member-side thread portion that is formed in the through hole,

the first member-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the second member includes a shaft portion that can be passed through the through hole of the first member and a head portion that is provided at a proximal end of the shaft portion and has an outer surface provided with a second member-side thread portion,

the second member-side thread portion can be screwed to the first thread groove of the first member-side thread portion.

The above object also can be achieved by the following.

A bone plate having a through hole for fixing a bone screw, the bone plate comprising:

a base plate, the through hole that extends through the base plate, and a plate-side thread portion that is formed in the through hole, wherein

the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the first thread groove of the plate-side thread portion is configured such that a thread portion formed in the bone screw can be screwed to the first thread groove.

The above object also can be achieved by the following.

A bone treatment tool comprising a bone plate and a bone screw, wherein

the bone plate includes a base plate, a through hole that extends through the base plate, and a plate-side thread portion that is formed in the through hole,

the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the bone screw includes a shaft portion that can be passed through the through hole of the bone plate and a head portion that is provided at a proximal end of the shaft portion and has an outer surface provided with a bone screw-side thread portion,

the bone screw-side thread portion can be screwed to the first thread groove of the plate-side thread portion.

### BRIEF DESCRIPTION OF THE DRAWINGS

FIG. 1 is a plan view showing an embodiment of a bone plate in which a fixation mechanism of the present invention can be used.

FIG. 2 is a side view showing an embodiment of a bone treatment tool including the bone plate shown in FIG. 1 in which the fixation mechanism of the present invention can be used.

FIG. 3 is an enlarged plan view showing a through hole portion of the bone plate shown in FIG. 1.

FIG. 4 is a cross-sectional view taken along line A-A in FIG. 3.

FIG. 5 is a cross-sectional view taken along line B-B in FIG. 3.

FIG. 6 is a cross-sectional view corresponding to FIG. 5 and showing a process of forming a plate-side thread portion in the bone plate shown in FIG. 1.

FIG. 7 is a cross-sectional view corresponding to FIG. 5 and showing the process of forming the plate-side thread portion in the bone plate shown in FIG. 1.

FIG. 8 is a front view showing an embodiment of a bone screw used in the bone treatment tool shown in FIG. 2.

FIG. 9 is a plan view of the bone screw shown in FIG. 8.

FIG. 10 is a cross-sectional view showing an example of the manner of fixing the bone screw to the bone plate in the bone treatment tool shown in FIG. 2.

FIG. 11 is a cross-sectional view showing another example of the manner of fixing the bone screw to the bone plate in the bone treatment tool shown in FIG. 2.

### EMBODIMENTS OF THE INVENTION

A fixation mechanism of the present invention will be described using an embodiment shown in the drawings. This fixing mechanism can also be called a fixing mechanism with selectable fixing angle.

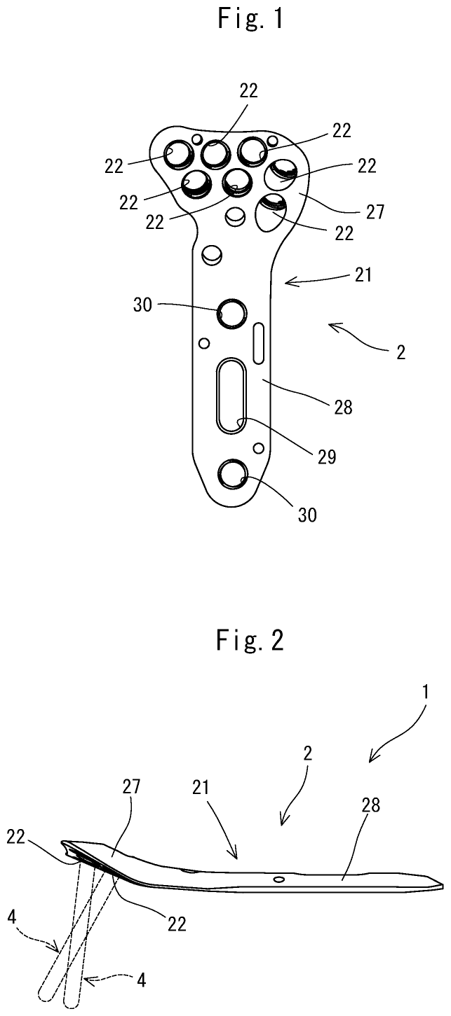

As shown in FIGS. 1 to 9, the fixation mechanism of the present embodiment is a fixation mechanism in which a first member 2 (bone plate 2) and a second member 4 (bone screw 4) can be fixed to each other at a suitable (any) angle. The first member 2 includes a base plate 21, through holes 22 that extend through the base plate 21, and a first member-side thread portion 23 (plate-side thread portion 23) that is formed in each through hole 22. In the first member-side thread portion 23, a first thread groove 24 that extends in the axial direction of the through hole 22 while turning in one turning direction is formed in the inner surface of the through hole 22, and a second thread groove 25 that extends in the axial direction of the through hole 22 while turning in a turning direction opposite to the one turning direction and intersects the first thread groove 24 at least one position, and the thread divided portion 26 being formed as a result of the first thread groove 24 and the second thread groove 25 intersecting each other. The second member 4 includes a shaft portion 41 that can be passed through the through hole 22 of the first member 2 and a head portion 42 that is provided at the proximal end of the shaft portion 41 and has an outer surface provided with a second member-side thread portion 43 (bone screw-side thread portion 43). The second member-side thread portion 43 can be screwed to the first thread groove 24 of the first member-side thread portion 23.

The second member-side thread portion 43 engages with the thread divided portion 26 when the second member 4 is rotated with the shaft portion 41 of the second member 4 penetrating the through hole 22 of the first member 2 at an angle at which the second member-side thread portion 43 and the first member-side thread portion 23 do not screw with the first thread groove 24, and the second member 4 is fixed so that it cannot move and cannot rotate in the axial direction of the through hole 22 at the angle.

The second member-side thread portion 43 can come into pressure contact with or engage with a thread divided portion 26. The second member 4 is fixed at a suitable (any) angle relative to the first member 2 so as not to be movable in the axial direction of the through hole 22 and not to be turnable, due to the pressure contact or engagement between the second member-side thread portion 43 and the thread divided portion 26.

When the second member-side thread portion 43 is screwed to the first thread groove 24 of the first member-side thread portion 23, the central axis of the first thread groove 24 matches the central axis of the second member-side thread portion 43 (external thread portion). The second member is fixed to the first member in a normal screwed state. When the second member-side thread portion 43 is in pressure contact with or engages with the thread divided portion 26, the second member 4 is fixed at a suitable (any) angle relative to the first member 2 so as not to be movable in the axial direction of the through hole 22 and not to be turnable. The second member is fixed to the first member in an inclined state through the pressure contact or engagement.

The fixation mechanism of the present invention can be used in a bone treatment tool 1 that includes a bone plate 2 and a bone screw 4. As shown in FIGS. 1 to 9, the bone treatment tool 1 in which the fixation mechanism of the present embodiment can be used includes the bone plate 2 (first member 2) and the bone screw 4 (second member 4). The bone plate 2 includes a base plate 21, through holes 22 that extend through the base plate 21, and a plate-side thread portion 23 (first member-side thread portion 23) that is formed in each through hole 22. In the plate-side thread portion 23, a first thread groove 24 that extends in the axial direction of the through hole 22 while turning in one turning direction is formed in the inner surface of the through hole 22, and a second thread groove 25 that extends in the axial direction of the through hole 22 while turning in a turning direction opposite to the one turning direction and intersects the first thread groove 24 at least one position, and the thread divided portion 26 being formed as a result of the first thread groove 24 and the second thread groove 25 intersecting each other. The bone screw 4 includes a shaft portion 41 that can be passed through the through hole 22 of the bone plate 2 and a head portion 42 that is provided at the proximal end of the shaft portion 41 and has an outer surface provided with a bone screw-side thread portion 43 (second member-side thread portion 43). The bone screw-side thread portion 43 can be screwed to the first thread groove 24 of the plate-side thread portion 23.

The bone screw-side thread portion 43 engages with the thread divided portion 26 when the bone screw 4 is rotated with the shaft portion 41 of the bone screw 4 penetrating the through hole 22 of the bone plate 2 at an angle at which the bone screw-side thread portion 43 and the plate-side thread portion 23 do not screw with the first thread groove 24, and the bone screw 4 is fixed so that it cannot move and cannot rotate in the axial direction of the through hole 22 at the angle.

The bone screw-side thread portion 43 can come into pressure contact with or engage with a thread divided portion 26. The bone screw 4 is fixed at a suitable (any) angle relative to the bone plate 2 so as not to be movable in the axial direction of the through hole 22 and not to be turnable, due to the pressure contact or engagement between the bone screw-side thread portion 43 and the thread divided portion 26.

When the bone screw-side thread portion 43 is screwed to the first thread groove 24 of the plate-side thread portion 23, the central axis of the first thread groove 24 matches the central axis of the bone screw-side thread portion 43 (external thread portion). The bone screw is fixed to the bone plate in a normal screwed state. When the bone screw-side thread portion 43 is in pressure contact with or engages with the thread divided portion 26, the bone screw 4 is fixed at a suitable (any) angle relative to the bone plate 2 so as not to be movable in the axial direction of the through hole 22 and not to be turnable. The bone screw is fixed to the bone plate in an inclined state through the pressure contact or engagement.

As shown in FIGS. 1 and 2, the base plate 21 of the bone plate 2 is formed into a T-shaped thin plate in a plan view. The base plate 21 includes a head portion 27 and a plate main body 28. The head portion 27 is joined to the plate main body 28 so as to be inclined, and the base plate 21 is bent at the boundary between the head portion 27 and the plate main body 28. Such a bone plate 2 is used for treating a fracture of the distal radius, for example.

The plate main body 28 of the bone plate 2 includes a fixation hole 29 that has a rounded rectangular shape in a plan view. The fixation hole 29 does not have an internal thread portion on its inner surface. In common procedures, the bone plate 2 is initially fixed to a target portion using the fixation hole 29 and a bone screw (which does not include a thread portion (external thread portion) on its head portion).

A plurality of (in this example, two) screw holes 30 for fixing bone screws are provided on two sides in the longitudinal direction of the fixation hole 29 in the plate main body 28. Internal thread portions are provided in inner surfaces of the screw holes 30. The internal thread portions are formed so as to extend parallel along axial directions of the screw holes 30. The internal thread portions are configured such that external thread portions provided in head portions of bone screws (not shown) can be screwed to the internal thread portions. The bone screws are fixedly attached to the bone plate 2 (plate main body 28) by being screwed to the internal thread portions.

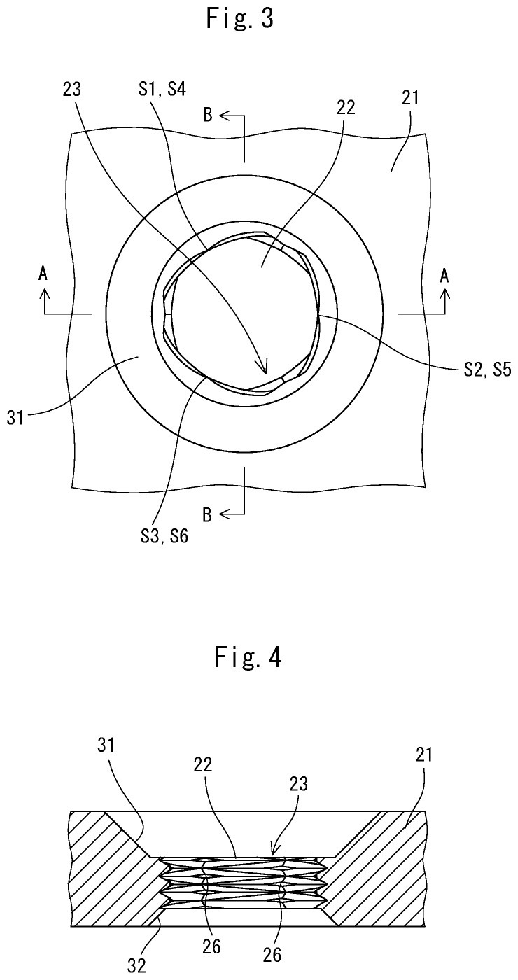

A plurality of (in this example, seven) through holes 22 are provided in the head portion 27 of the bone plate 2. As shown in FIGS. 3 to 5, these through holes 22 each have an inner diameter that is constant in the axial direction, and central axes of the through holes are inclined forming an angle relative to each other. An upper recessed portion 31 that spreads upward and a lower recessed portion 32 that spreads downward are respectively formed on the upper side and the lower side of each through hole 22. The upper recessed portion 31 and the lower recessed portion 32 can accommodate the entirety or a part of the head portion 42 of the bone screw 4, which will be described later. The plate-side thread portion 23 is formed in each of the through holes 22.

In the plate-side thread portion 23, the first thread groove 24 that extends in the axial direction of the through hole 22 while turning in one turning direction is formed in the inner surface of the through hole 22. In this example, the first thread groove 24 is constituted by helical grooves that form a so-called right-hand thread, which advances in the axial direction while turning clockwise in a plan view.

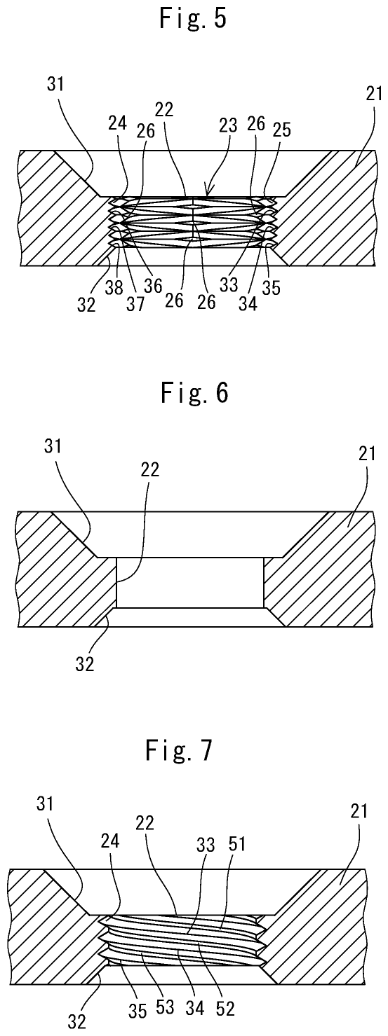

Specifically, as shown in FIG. 5, the first thread groove 24 is constituted by a plurality of helical grooves. In this example, the first thread groove 24 is constituted by three thread grooves (helical grooves) 33, 34, and 35. Accordingly, in the first thread groove 24, the lead (distance by which a screw advances in one turn): L1 is three times the pitch (distance between thread grooves (thread ridges) adjacent to each other in the axial direction): P1. The number of helical grooves constituting the first thread groove 24 is preferably 1 to 4, and particularly preferably 2 or 3.

In the plate-side thread portion 23, the three thread grooves 33, 34, and 35 constituting the first thread groove 24 have the same cross-sectional shape, which is a triangular shape in this example. The cross-sectional shape of the thread grooves 33, 34, and 35 is not limited to a triangular shape, and may be a trapezoidal shape or a rectangular shape, but a triangular shape and a trapezoidal shape are preferable for reasons described later. The first thread groove 24 constitutes a so-called triple-start thread, and the thread grooves 33, 34, and 35 constituting the first thread groove 24 have starting ends that are arranged at equal intervals in the circumferential direction (equal angles relative to the central axis of the through hole), specifically, at 120° intervals, in an upper end portion of the through hole 22 (in FIG. 3, the starting ends of the thread grooves 33, 34, and 35 are indicated by S1, S2, and S3, respectively).

Moreover, in the plate-side thread portion 23, the second thread groove 25 that extends in the axial direction of the through hole 22 while turning in the turning direction (counterclockwise direction) opposite to the turning direction (clockwise direction) of the above-described first thread groove 24 and intersects the first thread groove 24 at least one position is formed in the inner surface of the through hole 22. The second thread groove 25 is constituted by helical grooves that form a so-called left-hand thread, which advances in the axial direction while turning counterclockwise in a plan view. In the present embodiment, the central axis of the above-described first thread groove 24 (internal thread portion formed in the through hole 22 as a result of formation of the first thread groove 24) matches the central axis of the second thread groove 25 (internal thread portion formed in the through hole 22 as a result of formation of the second thread groove 25).

Specifically, as shown in FIG. 5, the second thread groove 25 is constituted by a plurality of helical grooves. In this example, the second thread groove 25 is constituted by three thread grooves (helical grooves) 36, 37, and 38. Accordingly, in the second thread groove 25, the lead (distance by which a screw advances in one turn): L2 is three times the pitch (distance between thread grooves (thread ridges) adjacent to each other in the axial direction): P2. The number of helical grooves constituting the second thread groove 25 is preferably 1 to 4, and particularly preferably 2 or 3.

In the plate-side thread portion 23, the three thread grooves 36, 37, and 38 constituting the second thread groove 25 have the same cross-sectional shape, which is a triangular shape in this example. The cross-sectional shape of the thread grooves 36, 37, and 38 is not limited to a triangular shape, and may be a trapezoidal shape or a rectangular shape, but a triangular shape and a trapezoidal shape are preferable for the reasons described later. The second thread groove 25 constitutes a so-called triple-start thread, and the thread grooves 36, 37, and 38 constituting the second thread groove 25 have starting ends S4, S5, and S6 that are arranged at equal intervals in the circumferential direction (equal angles relative to the central axis of the through hole), specifically, at 120° intervals, in the upper end portion of the through hole 22 (in FIG. 3, the starting ends of the thread grooves 36, 37, and 38 are indicated by S4, S5, and S6, respectively). In the present embodiment, the starting end S1 of the thread groove 33 is the same as the starting end S4 of the thread groove 36, the starting end S2 of the thread groove 34 is the same as the starting end S5 of the thread groove 37, and the starting end S3 of the thread groove 35 is the same as the starting end S6 of the thread groove 38. It should be noted that the positions of the starting ends S4, S5, and S6 of the thread grooves 36, 37, and 38 may be shifted from the positions of the starting ends S1, S2, and S3 of the thread grooves 33, 34, and 35 in the circumferential direction.

In the present embodiment, the turning directions of the first thread groove 24 and the second thread groove 25 are opposite to each other, and the first thread groove 24 and the second thread groove 25 have the same number of grooves, the same pitch, the same lead, and the same cross-sectional shape.

Furthermore, in the present embodiment, the inner diameter of each through hole 22 is constant in the axial direction as described above, and the first thread groove 24 and the second thread groove 25 are coaxially formed and both have a constant groove depth in the axial direction, and accordingly, the plate-side thread portion 23 of the bone plate 2 is a so-called parallel thread portion (straight thread portion).

As shown in FIGS. 4 and 5, in the bone plate 2, a plurality of thread divided portions 26 are formed as a result of the first thread groove 24 and the second thread groove 25 of the first member-side thread portion 23 intersecting each other in the through hole 22. That is, the thread divided portions 26 are formed as a result of thread ridges formed by forming the first thread groove 24 in the inner surface of the through hole 22 being divided by the second thread groove 25. In other words, the thread divided portions 26 can also be said to be portions remaining even after the second thread groove 25 has been formed, out of the thread ridges formed by forming the first thread groove 24 in the inner surface of the through hole 22. In the present embodiment, the first thread groove 24 and the second thread groove 25 have triangular cross-sectional shapes, and accordingly, the thickness (width) of each thread divided portion 26 in the axial direction of the through hole 22 decreases (the strength decreases) toward end portions of the thread divided portion 26 in the circumferential direction (turning direction).

Preferable examples of the material of the bone plate 2 include titanium alloys (specifically, Ti-6A1-4V in JIS T7401-2 and ASTM F-136 Ti-6A1-4V ELI), pure titanium (specifically, JIS T7401-1), and stainless steel (specifically, SUS304 and SUS316 in JIS G4303).

In order to explain the form of the thread divided portions 26 more specifically, a process of forming the plate-side thread portion 23 in the bone plate 2 as shown in FIGS. 5 to 7 will be described.

First, as shown in FIG. 6, a circular through hole 22 is formed as a pilot hole in the base plate 21 (head portion 27) of the bone plate 2. Next, as shown in FIG. 7, the first thread groove 24 (thread grooves 33, 34, and 35) is formed in the inner surface of the through hole 22. The first thread groove 24 can be formed through cutting using a known screw tap.

When the first thread groove 24 is formed, thread ridges 51, 52, and 53 are formed between adjacent thread grooves 33, 34, and 35. The cross-sectional shape of the thread ridges 51, 52, and 53 is a trapezoidal shape having an upper base at the inner circumferential surface of the through hole 22. At this point in time, it can be said that an internal thread portion including the thread grooves 33, 34, and 35 and the thread ridges 51, 52, and 53 is formed in the through hole 22. The first thread groove 24 (in other words, the internal thread portion formed in the through hole 22 as a result of formation of the first thread groove 24) is configured such that the bone screw-side thread portion 43 (external thread portion) of the bone screw 4, which will be described later, can be screwed to the first thread groove 24.

Next, as shown in FIG. 5, the second thread groove 25 (thread grooves 36, 37, and 38) is formed in the inner surface of the through hole 22. The second thread groove 25 can also be formed through cutting using a known screw tap. In the present embodiment, both the first thread groove 24 and the second thread groove 25 are formed so as to be coaxial with the through hole 22. Therefore, a processing axis (rotation axis of the screw tap used in the cutting processing) need not be changed when forming the first thread groove 24 and the second thread groove 25, and the processing can be easily performed. It should be noted that the first thread groove 24 and the second thread groove 25 can be formed using the same processing axis and reversing the rotation of the screw tap, in which case as well, the processing can be easily performed.

The second thread groove 25 intersects the first thread groove 24 at at least one position in the through hole 22. In the present embodiment, the first thread groove 24 and the second thread groove 25 intersect each other at a plurality of positions, and accordingly, a plurality of thread divided portions 26 are formed. In other words, the thread divided portions 26 are formed as a result of the thread ridges 51, 52, and 53 formed by forming the first thread groove 24 (thread grooves 33, 34, and 35) being divided by the thread grooves 36, 37, and 38 of the second thread groove 25. End portions of the thread divided portions 26 are exposed at intersections of the first thread groove 24 and the second thread groove 25. The thread divided portions 26 can also be said to be portions remaining in the through hole 22 (i.e., portions in which no thread groove is formed) when the first thread groove 24 and the second thread groove 25 are formed.

In the present embodiment, the first thread groove 24 and the second thread groove 25 are formed so as to have triangular cross-sectional shapes. Accordingly, the thickness (width) of each thread divided portion 26 in the axial direction of the through hole 22 decreases (the strength decreases) toward end portions of the thread divided portion 26 in the circumferential direction (turning direction). In other words, the thickness (width) of the thread divided portions 26 decreases toward positions where the first thread groove 24 and the second thread groove 25 intersect each other. Such a form can be realized in a case where the cross-sectional shape of the first thread groove 24 and/or the second thread groove 25 is a triangular shape or a trapezoidal shape, and therefore, it is preferable that the cross-sectional shape of the first thread groove 24 and/ or the second thread groove 25 is a triangular shape or a trapezoidal shape.

It should be noted that the first thread groove 24 and the second thread groove 25 do not necessarily have to be formed (processed) through cutting using a known screw tap as described above, and may be formed through rolling, turning, or a combination of any of rolling, turning, and cutting, for example. Moreover, in the present embodiment, the first thread groove 24 is initially formed in the inner surface of the through hole 22 and thereafter the second thread groove 25 is formed, in order to facilitate understanding of the process of forming the thread divided portions 26, but it is also possible to initially form the second thread groove 25. In this case as well, the thread ridges 51, 52, and 53 formed by forming the first thread groove 24 are divided at positions where the second thread groove 25 has been formed in advance, and finally the plate-side thread portion 23 including the thread divided portions 26 has the same shape.

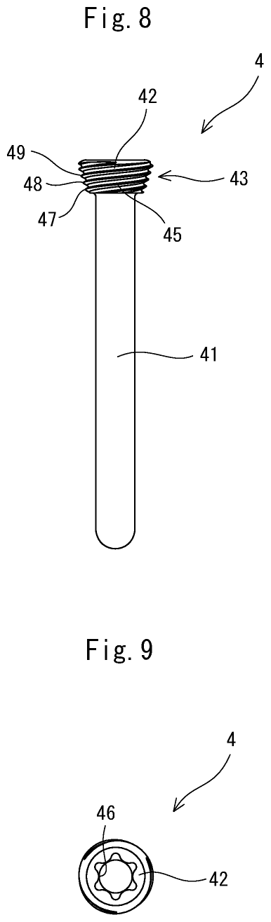

As shown in FIGS. 8 and 9, the bone screw 4 used in the present embodiment includes the shaft portion 41 and the head portion 42. The shaft portion 41 has a smooth surface and can be passed through the through hole 22 of the bone plate 2. Also, the shaft portion 41 can enter a bone (a pilot hole formed in the target bone), and the outer diameter of the shaft portion 41 depends on the treatment target portion, but is preferably 2.0 mm to 7.5 mm, and particularly preferably 2.5 mm to 4.0 mm.

The head portion 42 of the bone screw 4 includes a tapered portion 45 that is continuous to the proximal end of the shaft portion 41. The diameter of the tapered portion 45 decreases toward the distal end side (shaft portion 41 side). Also, as shown in FIG. 9, the head portion 42 includes a recessed portion 46 for connecting a rotating jig (e.g., a screwdriver). The recessed portion 46 is formed into a shape that corresponds to the shape of the tip end of the rotating jig.

The head portion 42 of the bone screw 4 is provided with the bone screw-side thread portion 43 formed on the outer surface. In the present embodiment, the bone screw-side thread portion 43 is formed in the tapered portion 45 of the head portion 42. The bone screw-side thread portion 43 is constituted by helical ridges (thread ridges) that form a so-called right-hand thread, which advances in the axial direction while turning clockwise in a plan view (when viewed from the head portion 42 side in the axial direction of the bone screw 4). The bone screw-side thread portion 43 is a tapered thread portion formed in the tapered portion 45 that is formed in the head portion 42 of the bone screw 4 and of which the diameter decreases toward the distal end side. The bone screw-side thread portion 43 can be screwed to the first thread groove 24 of the plate-side thread portion 23 of the bone plate 2. In other words, the bone screw-side thread portion 43 can enter or be screwed to the internal thread portion formed in the plate-side thread portion 23 by forming the first thread groove 24, and when the bone screw 4 is rotated (in this example, rotated clockwise in a plan view) in a state where the axial center (central axis) of the bone screw 4 (bone screw-side thread portion 43) substantially matches the axial center (central axis) of the through hole 22 of the bone plate 2, the bone screw-side thread portion 43 enters or is screwed to the first thread groove 24 of the plate-side thread portion 23.

More specifically, as shown in FIG. 8, the bone screw-side thread portion 43 includes a plurality of (in this example, three) thread ridges 47, 48, and 49. Accordingly, in the bone screw-side thread portion 43, the lead (distance by which the screw advances in one turn): L3 is three times the pitch (distance between thread ridges (thread grooves) adjacent to each other in the axial direction): P3. The number of ridges constituting the bone screw-side thread portion 43 is preferably the same as the number of helical grooves constituting the first thread groove 24, and is preferably 1 to 4, and particularly preferably 2 or 3.

The three thread ridges 47, 48, and 49 constituting the bone screw-side thread portion 43 have the same cross-sectional shape, which is a triangular shape corresponding to the cross-sectional shape of the first thread groove 24 (thread grooves 33, 34, and 35) of the plate-side thread portion 23 in this example. The cross-sectional shape of the thread ridges 47, 48, and 49 is not limited to a triangular shape, and may be a trapezoidal shape or a rectangular shape, but a triangular shape and a trapezoidal shape are preferable. The thread ridges 47, 48, and 49 constituting the bone screw-side thread portion 43 have starting ends that are arranged at equal intervals in the circumferential direction (equal angles relative to the central axis of the bone screw), specifically, at 120° intervals, in a distal end side portion of the bone screw-side thread portion 43 (portion joined to the shaft portion 41).

As in the case of the bone plate 2, preferable examples of the material of the bone screw 4 include titanium alloys (specifically, Ti-6A1-4V in JIS T7401-2 and ASTM F-136 Ti-6A1-4V ELI), pure titanium (specifically, JIS T7401-1), and stainless steel (specifically, SUS304 and SUS316 in JIS G4303).

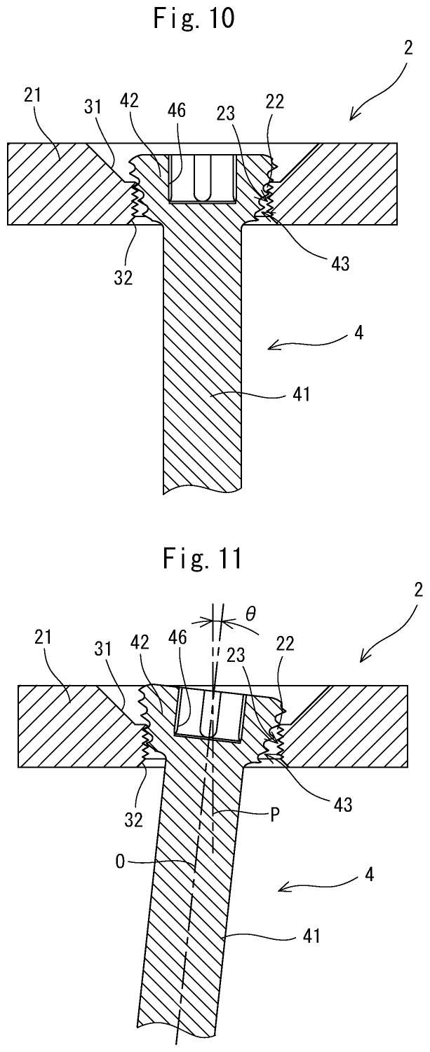

Next, the manner of fixing the bone screw 4 to the bone plate 2 will be described. As shown in FIG. 10, the bone screw-side thread portion 43 enters or is screwed to the first thread groove 24 of the plate-side thread portion 23 as a result of the bone screw 4 being rotated (in this example, rotated clockwise in a plan view) in the state where the axial center (central axis) of the bone screw 4 (bone screw-side thread portion 43) substantially matches the axial center (central axis) of the through hole 22 of the bone plate 2. In the present embodiment, the bone screw-side thread portion 43 is a tapered thread portion, and accordingly, as the bone screw-side thread portion 43 advances into the first thread groove 24, the bone screw-side thread portion 43 engages with (is screwed to) the first thread groove 24 of the plate-side thread portion 23. By screwing the bone screw-side thread portion 43 to the first thread groove 24 of the plate-side thread portion 23 as described above, the bone screw 4 can be fixed at an angle (0° with respect to the axial direction of the through hole 22) relative to the bone plate 2 so as to extend in the axial direction of the through hole 22 and so as not to be movable in the axial direction of the through hole 22 and not to be turnable.

Also, as shown in FIG. 11, when the bone screw 4 is inserted at a suitable (any) angle θ relative to the bone plate 2 (θ represents an angle formed between the axial center: P of the through hole 22 and the axial center: O of the bone screw 4), the bone screw-side thread portion 43 comes into pressure contact with or engages with the thread divided portions 26 formed as a result of the first thread groove 24 and the second thread groove 25 of the plate-side thread portion 23 intersecting each other, and accordingly, the bone screw 4 can be fixed at the suitable (any) angle relative to the bone plate 2 so as not to be movable in the axial direction of the through hole 22 and not to be turnable. Here, it is preferable that the bone screw 4 can be fixed to the bone plate 2 at an angle of 0° to 15° with respect to the axial direction of the through hole 22.

More specifically, the first thread groove 24 and the second thread groove 25 intersect each other in the through hole 22 of the bone plate 2, whereby the thread divided portions 26 are formed. Therefore, when the bone screw 4 is inserted in a state of being inclined at a suitable (any) angle relative to the bone plate 2, the distal end side portion of the head portion 42 (tapered portion 45) of the bone screw 4, which has a relatively small diameter, enters the through hole 22, and as the bone screw 4 advances further, the bone screw-side thread portion 43 (thread ridges 47, 48, and 49) enters an intersection of the first thread groove 24 and the second thread groove 25, and comes into pressure contact with or engages with a thread divided portion 26 from an end portion side of the thread divided portion 26.

As described above, in the present embodiment, the thickness (width) of the thread divided portion 26 in the axial direction of the through hole 22 decreases (the strength decreases) toward end portions of the thread divided portion 26 in the circumferential direction (turning direction). Accordingly, when the bone screw-side thread portion 43 (thread ridges 47, 48, and 49) enters the intersection of the first thread groove 24 and the second thread groove 25 and comes into contact with the end portion of the thread divided portion 26, a resistance force (a reaction force generated due to the bone screw-side thread portion 43 coming into contact with the thread divided portion 26) is initially small, but gradually increases, and the bone screw-side thread portion 43 finally reaches the state of pressure contact or engagement. Therefore, when the bone screw-side thread portion 43 comes into contact with the thread divided portion 26, the resistance force keeps the insertion angle of the bone screw 4 from shifting, and the insertion angle of the bone screw 4 can be adjusted more precisely. It should be noted that plastic deformation (deformation in a range exceeding elastic deformation) of the thread divided portion 26 may occur in the process of the bone screw-side thread portion 43 coming into pressure contact with or engaging with the thread divided portion 26.

Furthermore, the upper recessed portion 31 that spreads upward and the lower recessed portion 32 that spreads downward are respectively formed on the upper side and the lower side of the through hole 22 of the bone plate 2. Therefore, even when the bone screw 4 is inserted in the state of being inclined at a suitable (any) angle relative to the bone plate 2, the head portion 42 of the bone screw 4 is entirely or partially housed in the upper recessed portion 31 and the lower recessed portion 32, and is kept from protruding from the upper surface and the lower surface of the bone plate 2.

It is known that, in a living body, the behavior of a screw product for orthopedic surgery under an envisaged load varies depending on whether the screw is a right-hand screw or a left-hand screw. That is, depending on the portion to which the bone screw is applied or the insertion direction of the bone screw, there are cases where a specific turning direction is appropriate for preventing loosening or backing out (pulling out) of the bone screw. Therefore, two types of (right-hand and left-hand) bone screws that differ from each other only in the turning direction of the thread portion (external thread portion) of the head portion are sometimes included in a bone treatment operation set including bone plates and bone screws.

Under the above circumstances, the bone plate 2 of the present embodiment can be used with both bone screws having opposite turning directions. That is, in the bone plate 2 of the present embodiment, the first thread groove 24 and the second thread groove 25 differ from each other only in the turning direction (the first thread groove 24 is a right-hand thread and the second thread groove 25 is a left-hand thread), and have the same number of grooves, the same pitch, the same lead, and the same cross-sectional shape. Therefore, when a right-hand bone screw is used, the head portion (bone screw-side thread portion) of the bone screw enters the first thread groove 24, and is screwed to the first thread groove 24 or comes into pressure contact with or engage with the thread divided portion 26. On the other hand, when a left-hand bone screw is used, the head portion (bone screw-side thread portion) of the bone screw enters the second thread groove 25, and is screwed to the second thread groove 25 or comes into pressure contact with or engages with the thread divided portion 26.

That is, in the bone treatment tool (1) in which the fixation mechanism of the present embodiment can be used, may further includes a third member (left-hand bone screw) that can be fixed to the first member (bone plate 2) in the through hole (22) of the first member (2). The third member includes a shaft portion that can be passed through the through hole (22) of the first member (2), a head portion provided at a proximal end of the shaft portion and a third member-side thread portion (left-hand thread portion) provided at the head portion. The third member-side thread portion can be screwed to the second thread groove (25) of the through hole (22).

The third member-side thread portion engages with the thread divided portion (26) when the third member is rotated at an angle at which the third member-side thread portion and the first member-side thread portion (plate-side thread portion 23) do not screw with the second thread groove (25), and the third member is fixed so that it cannot move and cannot rotate in the axial direction of the through hole (22) at the angle.

The third member-side thread portion can come into pressure contact with or engage with the thread divided portion (26), and the third member is fixed at a suitable (any) angle relative to the first member so as not to be movable in the axial direction of the through hole and not to be turnable, due to the pressure contact or engagement between the third member-side thread portion and the thread divided portion.

When the third member-side thread portion is screwed to the second thread groove (25) of the first member-side thread portion (23), the central axis of the second thread groove (25) matches the central axis of the third member-side thread portion (external thread portion). The third member is fixed to the first member in a normal screwed state. When the third member-side thread portion is in pressure contact with or engages with the thread divided portion (26), the third member is fixed at a suitable (any) angle relative to the first member (2) so as not to be movable in the axial direction of the through hole (22) and not to be turnable. The third member is fixed to the first member in an inclined state through the pressure contact or engagement.

It should be noted that the first thread groove and the second thread groove in the bone plate (first member) may differ from each other in the number of grooves, the pitch, the lead, or the cross-sectional shape. In this case, it is preferable that a ratio P2/ P1 between the pitch: P2 of the second thread groove and the pitch: P1 of the first thread groove is 0.5 to 1.5.

Also, a plurality of thread grooves may be formed as thread grooves having a turning direction that differs from the turning direction of the first thread groove. In this case, thread ridges formed by forming the first thread groove are divided by those thread grooves, and as a result of end portions of the thus formed thread divided portions coming into pressure contact with or engaging with the second member-side thread portion, the second member can be fixed at a suitable (any) angle relative to the first member.

The second thread groove may be constituted by a groove that does not form a common thread and merely extends helically in the axial direction of the through hole. In this case as well, the first thread groove and the second thread groove intersect each other at at least one position, and accordingly, an end portion of a thread divided portion is formed at the position where a thread ridge formed by forming the first thread groove is divided by the second thread groove.

In the present embodiment, the first thread groove 24 is constituted by the helical grooves forming a so-called right-hand thread that extends in the axial direction while turning clockwise in a plan view, but the first thread groove may be constituted by helical grooves forming a so-called left-hand thread that extends in the axial direction while turning counterclockwise in a plan view.

The first member-side thread portion (plate-side thread portion) may be formed as a tapered thread portion, and the second member-side thread portion (bone screw-side thread portion) may be formed as a straight thread portion. It should be noted that even in a case where both the first member-side thread portion and the second member-side thread portion are straight thread portions and the second member (bone screw) is inserted in a state where the axial center of the second member substantially matches the axial center of the through hole of the first member (bone plate), the first member and the second member engage (are screwed) so as not to be movable in the axial direction and not to be turnable at incomplete thread portions that are inevitably formed at end portions of the first member-side thread portion and the second member-side thread portion.

In the present embodiment, the bone screw including the shaft portion having a smooth surface is used, but it is also possible to use a bone screw including a shaft portion in which a thread portion (tapping thread portion) is formed. In this case, it is preferable that the thread portion formed in the shaft portion can pass through the through hole of the bone plate so as not to interfere with the plate-side thread portion formed in the inner surface of the through hole.

In the above-described embodiment, the fixation mechanism of the present invention, and the bone treatment tool and the bone plate in which the fixation mechanism can be used are described using the bone treatment tool 1 and the bone plate 2 for the distal radius as examples. The fixation mechanism of the present invention can be used not only in the bone treatment tool and the bone plate described above, but also in bone treatment tools for CHS (for fractures in the proximal part of a femur), the vertebral column, fingers, toes, tooth formation, and artificial joints.

The fixation mechanism of the present invention can also be used in the architectural field and the like, in addition to the medical field. For example, when attaching a plate-like member to a relatively soft member (resin member) in construction-related work, a pressure force can be increased by inserting a screw (tapping screw) diagonally. However, it is troublesome to form screw holes having different insertion angles in the plate-like member in advance. Also, when a screw for metal is used, it may be necessary to devise a way to increase the pullout strength required to pull the screw out of the resin member. When the fixation mechanism of the present invention is used, the screw can be inserted at a suitable (any) angle without the need to vary the formation angle of the screw hole, and furthermore, the head portion of the screw can be fixed in the screw hole, and therefore, it is advantageous to use the fixation mechanism of the present invention.

The following is an aspect of a fixation mechanism of the present invention.
(1) A fixation mechanism that can fix a first member and a second member to each other at a suitable angle, wherein

the first member includes a base plate, a through hole that extends through the base plate, and a first member-side thread portion that is formed in the through hole,

the first member-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the second member includes a shaft portion that can be passed through the through hole of the first member and a head portion that is provided at a proximal end of the shaft portion and has an outer surface provided with a second member-side thread portion,

the second member-side thread portion can be screwed to the first thread groove of the first member-side thread portion.

In the fixation mechanism of the present invention, the first member includes the base plate portion, the through hole that extends through the base plate portion, and the first member-side thread portion that is formed in the through hole. In the first member-side thread portion, the first thread groove that extends in the axial direction of the through hole while turning in one turning direction is formed in the inner surface of the through hole, and the second thread groove that extends in the axial direction of the through hole while turning in a turning direction opposite to the one turning direction and intersects the first thread groove at at least one position is formed in the inner surface of the through hole. The second member includes the shaft portion that can be passed through the through hole of the first member and the head portion that is provided at the proximal end of the shaft portion and has the outer surface provided with the second member-side thread portion. The second member-side thread portion can be screwed to the first thread groove of the first member-side thread portion.

Therefore, by screwing the second member-side thread portion into the first thread groove of the first member-side thread portion, the second member can be fixed at an angle (0° with respect to the axial direction of the through hole) relative to the first member so as to extend in the axial direction of the through hole and so as not to be movable in the axial direction of the through hole and not to be turnable.

Furthermore, the second member-side thread portion can come into pressure contact with or engage with the thread divided portion that is formed as a result of the first thread groove and the second thread groove of the first member-side thread portion intersecting each other. Therefore, when the second member is inserted in a state of being inclined at a suitable (any) angle (e.g., up to about 15° with respect to the axial direction of the through hole) relative to the first member, the second member can be fixed at the suitable (any) angle relative to the first member so as not to be movable in the axial direction of the through hole and not to be turnable, due to the second member-side thread portion being in pressure contact with or engaging with the thread divided portion formed as a result of the first thread groove and the second thread groove of the first member-side thread portion intersecting each other (in other words, a portion formed as a result of a thread ridge formed on the inner surface of the through hole by forming the first thread groove being divided by the second thread groove).

The above-described aspect may be configured as described below.
(2) The fixation mechanism according to above (1), wherein the second member-side thread portion engages with the thread divided portion when the second member is rotated with the shaft portion of the second member penetrating the through hole of the first member at an angle at which the second member-side thread portion and the first member-side thread portion do not screw with the first thread groove, and the second member is fixed so that it cannot move and cannot rotate in the axial direction of the through hole at the angle.
(3) The fixation mechanism according to above (1) or (2), wherein the second member-side thread portion can come into pressure contact with or engage with the thread divided portion and the second member is fixed at a suitable angle relative to the first member so as not to be movable in the axial direction of the through hole and not to be turnable, due to pressure contact or engagement between the second member-side thread portion and the thread divided portion.
(4) The fixation mechanism according to any one of above (1) to (3), wherein the thickness of the thread divided portion decreases toward an end portion of the thread divided portion.
(5) The angle fixation mechanism according to any one of above (1) to (4), wherein the through hole has a constant inner diameter of in the axial direction,

the first thread groove and the second thread groove are coaxially formed and both have a constant groove depth in the axial direction, and

the second member has a tapered portion that is formed in the head portion of the second member and of which the diameter decreases toward a distal end side, and the second member-side thread portion is provided at the tapered portion.
(6) The fixation mechanism according to any one of above (1) to (5), wherein the first thread groove is constituted by a plurality of helical grooves and the second thread groove is constituted by a plurality of helical grooves.
(7) The fixation mechanism according to any one of above (1) to (6), wherein a ratio: P2/P1 between a pitch: P2 of the second thread groove and a pitch: P1 of the first thread groove is 0.5 to 1.5.
(8) The fixation mechanism according to any one of above (1) to (7), wherein turning directions of the first thread groove and the second thread groove are opposite to each other, and the first thread groove and the second thread groove have the same number of grooves, the same pitch, the same lead, and the same cross-sectional shape.
(9) The fixation mechanism according to any one of above (1) to (8), wherein the fixing mechanism is capable of fixing the second member at an angle of 0° to 15° with respect to the central axis of the through hole of the first member.
(10) The fixation mechanism according to any one of above (1) to (9), further includes a third member that can be fixed to the first member in the through hole of the first member, the third member includes a shaft portion that can be passed through the through hole of the first member, a head portion provided at a proximal end of the shaft portion and a third member-side thread portion provided at the head portion, the third member-side thread portion can be screwed to the second thread groove of the through hole.
(11) The fixation mechanism according to above (10), wherein the third member-side threaded portion engages with the thread divided portion when the third member is rotated with the shaft portion of the third member penetrating the through hole of the first member at an angle at which the third member-side thread portion and the first member-side thread portion do not screw with the second thread groove, and the third member is fixed so that it cannot move and cannot rotate in the axial direction of the through hole at the angle.
(12) The fixation mechanism according to above (10) or (11), wherein the third member-side thread portion can come into pressure contact with or engage with the thread divided portion and the third member is fixed at a suitable angle relative to the first member so as not to be movable in the axial direction of the through hole and not to be turnable, due to pressure contact or engagement between the third member-side thread portion and the thread divided portion.

The following is an aspect of a bone plate of the present invention.
(13) A bone plate having a through hole for fixing a bone screw, the bone plate comprising:

a base plate, the through hole that extends through the base plate, and a plate-side thread portion that is formed in the through hole, wherein

the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the first thread groove of the plate-side thread portion is configured such that a thread portion formed in the bone screw can be screwed to the first thread groove.

The above-described aspect may be configured as described below.
(14) The bone plate according to above (13), wherein the thread portion of the bone screw engages with the thread divided portion when the bone screw is rotated at an angle at which the thread portion of the bone screw and the plate-side thread portion do not screw with the first thread groove, and the bone screw is fixed so that it cannot move and cannot rotate in the axial direction of the through hole at the angle.
(15) The bone plate according to above (13) or (14), wherein the thread portion of the bone screw can come into pressure contact with or engage with the thread divided portion and the bone screw is fixed at a suitable angle relative to the bone plate so as not to be movable in the axial direction of the through hole and not to be turnable, due to pressure contact or engagement between the thread portion of the bone screw and the thread divided portion.

The following is an aspect of a bone treatment tool of the present invention.
(16) A bone treatment tool comprising a bone plate and a bone screw, wherein

the bone plate includes a base plate, a through hole that extends through the base plate, and a plate-side thread portion that is formed in the through hole,

the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,

the bone screw includes a shaft portion that can be passed through the through hole of the bone plate and a head portion that is provided at a proximal end of the shaft portion and has an outer surface provided with a bone screw-side thread portion,

the bone screw-side thread portion can be screwed to the first thread groove of the plate-side thread portion.

The above-described aspect may be configured as described below.
(17) The bone treatment tool according to above (16), wherein the bone screw-side thread portion engages with the thread divided portion when the bone screw is rotated at an angle at which the bone screw-side thread portion and the plate-side thread portion do not screw with the first thread groove, and the bone screw is fixed so that it cannot move and cannot rotate in the axial direction of the through hole at the angle.
(18) The bone treatment tool according to above (16) or (17), wherein the bone screw-side thread portion can come into pressure contact with or engage with the thread divided portion and the bone screw is fixed at a suitable angle relative to the bone plate so as not to be movable in the axial direction of the through hole and not to be turnable, due to pressure contact or engagement between the bone screw-side thread portion and the thread divided portion.
(19) The bone treatment tool according to any one of above (16) to (18), further includes a second bone screw that can be fixed to the bone plate in the through hole of the bone plate, the second bone screw includes a shaft portion that can be passed through the through hole of the bone plate, a head portion provided at a proximal end of the shaft portion and a third thread portion provided at the head portion, the third thread portion can be screwed to the second thread groove of the through hole.
(20) The bone treatment tool according to above (19), wherein the third thread portion engages with the thread divided portion when the second bone screw is rotated at an angle at which the third thread portion and the plate-side thread portion do not screw with the second thread groove, and the second bone screw is fixed so that it cannot move and cannot rotate in the axial direction of the through hole at the angle.
(21) The bone treatment tool according to above (19) or (20), wherein the third thread portion can come into pressure contact with or engage with the thread divided portion and the second bone screw is fixed at a suitable angle relative to the bone plate so as not to be movable in the axial direction of the through hole and not to be turnable, due to pressure contact or engagement between the third thread portion and the thread divided portion.

What is claimed is:

1. A fixation mechanism that can fix a first member and a second member to
each other at a suitable angle, wherein
the first member includes a base plate, a through hole that extends through the base plate, and a first member-side thread portion that is formed in the through hole,
the first member-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,
the second member includes a shaft portion that can be passed through the through hole of the first member and a head portion that is provided at a proximal end of the shaft portion and has an outer surface provided with a second member-side thread portion,
the second member-side thread portion can be screwed to the first thread groove of the first member-side thread portion.

2. The fixation mechanism according to claim 1, wherein the second member-side thread portion engages with the thread divided portion when the second member is rotated with the shaft portion of the second member penetrating the through hole of the first member at an angle at which the second member-side thread portion and the first member-side thread portion do not screw with the first thread groove, and the second member is fixed so that it cannot move and cannot rotate in the axial direction of the through hole at the angle.

3. The fixation mechanism according to claim 1, wherein the second member-side thread portion can come into pressure contact with or engage with the thread divided portion and the second member is fixed at a suitable angle relative to the first member so as not to be movable in the axial direction of the through hole and not to be turnable, due to pressure contact or engagement between the second member-side thread portion and the thread divided portion.

4. The fixation mechanism according to claim 1, wherein the thickness of the thread divided portion decreases toward an end portion of the thread divided portion.

5. The angle fixation mechanism according to claim 1, wherein the through hole has a constant inner diameter of in the axial direction,
the first thread groove and the second thread groove are coaxially formed and both have a constant groove depth in the axial direction, and
the second member has a tapered portion that is formed in the head portion of the second member and of which the diameter decreases toward a distal end side, and the second member-side thread portion is provided at the tapered portion.

6. The fixation mechanism according to claim 1, wherein the first thread groove is constituted by a plurality of helical grooves and the second thread groove is constituted by a plurality of helical grooves.

7. The fixation mechanism according to claim 1, wherein a ratio: P2/P1 between a pitch: P2 of the second thread groove and a pitch: P1 of the first thread groove is 0.5 to 1.5.

8. The fixation mechanism according to claim 1, wherein turning directions of the first thread groove and the second thread groove are opposite to each other, and the first thread groove and the second thread groove have the same number of grooves, the same pitch, the same lead, and the same cross-sectional shape.

9. The fixation mechanism according to claim 1, wherein the fixing mechanism is capable of fixing the second member at an angle of 0° to 15° with respect to the central axis of the through hole of the first member.

10. The fixation mechanism according to claim 1, further includes a third member that can be fixed to the first member in the through hole of the first member, the third member includes a shaft portion that can be passed through the through hole of the first member, a head portion provided at a proximal end of the shaft portion and a third member-side thread portion provided at the head portion, the third member-side thread portion can be screwed to the second thread groove of the through hole.

11. The fixation mechanism according to 10, wherein the third member-side threaded portion engages with the thread divided portion when the third member is rotated with the shaft portion of the third member penetrating the through hole of the first member at an angle at which the third member-side thread portion and the first member-side thread portion do not screw with the second thread groove, and the third member is fixed so that it cannot move and cannot rotate in the axial direction of the through hole at the angle.

12. The fixation mechanism according to claim 10, wherein the third member-side thread portion can come into pressure contact with or engage with the thread divided portion and the third member is fixed at a suitable angle relative to the first member so as not to be movable in the axial direction of the through hole and not to be turnable, due to pressure contact or engagement between the third member-side thread portion and the thread divided portion.

13. A bone plate having a through hole for fixing a bone screw, the bone plate comprising:
a base plate, the through hole that extends through the base plate, and a plate-side thread portion that is formed in the through hole, wherein
the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,
the first thread groove of the plate-side thread portion is configured such that a thread portion formed in the bone screw can be screwed to the first thread groove.

14. The bone plate according to claim 13, wherein the thread portion of the bone screw engages with the thread divided portion when the bone screw is rotated at an angle at which the thread portion of the bone screw and the plate-side thread portion do not screw with the first thread groove, and the bone screw is fixed so that it cannot move and cannot rotate in the axial direction of the through hole at the angle.

15. The bone plate according to claim 13, wherein the thread portion of the bone screw can come into pressure contact with or engage with the thread divided portion and the bone screw is fixed at a suitable angle relative to the bone plate so as not to be movable in the axial direction of the through hole and not to be turnable, due to pressure contact or engagement between the thread portion of the bone screw and the thread divided portion.

16. A bone treatment tool comprising a bone plate and a bone screw, wherein
the bone plate includes a base plate, a through hole that extends through the base plate, and a plate-side thread portion that is formed in the through hole,
the plate-side thread portion includes a first thread groove, a second thread groove, and a thread divided portion, the first thread groove being formed in an inner surface of the through hole and extending in an axial direction of the through hole while turning in one turning direction, the second thread groove being formed in the inner surface of the through hole, extending in the axial direction of the through hole while turning in a turning direction opposite to the one turning direction, and intersecting the first thread groove at least one position, and the thread divided portion being formed as a result of the first thread groove and the second thread groove intersecting each other,
the bone screw includes a shaft portion that can be passed through the through hole of the bone plate and a head portion that is provided at a proximal end of the shaft portion and has an outer surface provided with a bone screw-side thread portion,
the bone screw-side thread portion can be screwed to the first thread groove of the plate-side thread portion.

17. The bone treatment tool according to claim 16, wherein the bone screw-side thread portion engages with the thread divided portion when the bone screw is rotated at an angle at which the bone screw-side thread portion and the plate-side thread portion do not screw with the first thread groove, and the bone screw is fixed so that it cannot move and cannot rotate in the axial direction of the through hole at the angle.

18. The bone treatment tool according to claim 16, wherein the bone screw-side thread portion can come into pressure contact with or engage with the thread divided portion and the bone screw is fixed at a suitable angle relative to the bone plate so as not to be movable in the axial direction of the through hole and not to be turnable, due to pressure contact or engagement between the bone screw-side thread portion and the thread divided portion.

19. The bone treatment tool according to claim 16, further includes a second bone screw that can be fixed to the bone plate in the through hole of the bone plate, the second bone screw includes a shaft portion that can be passed through the through hole of the bone plate, a head portion provided at a proximal end of the shaft portion and a third thread portion provided at the head portion, the third thread portion can be screwed to the second thread groove of the through hole.
